(19) United States
(12) Patent Application Publication
(10) Pub. No.: US 2025/0270156 A1
(43) Pub. Date: Aug. 28, 2025

(54) METHOD FOR PRODUCING 3-CHLORO-1,1,1,5,5,5-HEXAFLUORO-2-PENTENE

(71) Applicant: KANTO DENKA KOGYO CO., LTD., Tokyo (JP)

(72) Inventors: Tomohiro TANIGUCHI, Okayama (JP); Masataka MIURA, Okayama (JP)

(21) Appl. No.: 18/859,912

(22) PCT Filed: Apr. 27, 2023
(86) PCT No.: PCT/JP2023/016592

§ 371 Date: Oct. 24, 2024

(30) Foreign Application Priority Data
Apr. 28, 2022 (JP) 2022-074964

(51) Int. Cl. C07C 17/20

(52) U.S. Cl. CPC C07C 17/206; C07C 2521/04; C07C 2523/06; C07C 2523/26; C07C 2527/132; C07C 2527/133; C07C 2527/135; C07C 2527/20; C07C 2529/06

(57) ABSTRACT

An object of the present invention is to provide a novel method for producing 3-chloro-1,1,1,5,5,5-hexafluoro-2-pentene. Provided is a method for producing 3-chloro-1,1,1,5,5,5-hexafluoro-2-pentene, comprising a step of reacting 1,1,1,3,3,5,5,5-octachloropentane with hydrogen fluoride. Also provided are a method for producing 3-chloro-1,1,1,5,5,5-hexafluoro-2-pentene, comprising a step of reacting 1,1,3-trichloro-5,5,5-trifluoro-1,3-pentadiene with hydrogen fluoride, and a method for producing 3-chloro-1,1,1,5,5,5-hexafluoro-2-pentene, comprising a step of reacting 1,1,3,5,5,5-hexachloro-1,3-pentadiene with hydrogen fluoride.

### TECHNICAL FIELD

The present invention relates to a method for producing 3-chloro-1,1,1,5,5,5-hexafluoro-2-pentene (hereinafter sometimes referred to as “HCFO-1446”).

### BACKGROUND ART

Fluoroolefins are used as solvents, cleaning agents, foaming agents, and intermediates for functional materials, and others, and various production methods thereof have been proposed. In particular, 3-chloro-1,1,1,5,5,5-hexafluoro-2-pentene is useful as a synthetic intermediate because it has, in its molecule, a C═C double bond and a chlorine substituent, which serve as reaction initiation points. For example, PTL 1 disclose, in Examples 1 and 2, that the fluorine at position 3 of 1,1,1,3,3,5,5,5-octafluoropentane (HFC-458) was chlorinated to produce 3,3-dichloro-1,1,1,5,5,5-hexafluoropentane (HFC-456), and HCl was then eliminated to produce 3-chloro-1,1,1,5,5,5-hexafluoro-2-pentene (HCFO-1446), as shown in the following reaction formula.

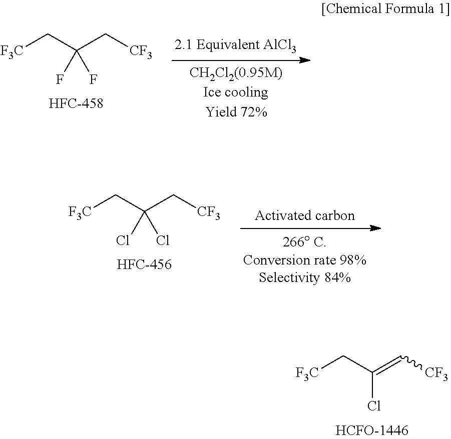 [SMILES: FC(F)(F)CC(F)(F)CC(F)(F)F ; FC(F)(F)CC(Cl)(Cl)CC(F)(F)F ; FC(F)(F)C=C(Cl)CC(F)(F)F]

However, there have been no reports that 3-chloro-1,1,1,5,5,5-hexafluoro-2-pentene was produced by a halogen exchange reaction of chlorine.

### CITATION LIST

### Patent Literature

PTL 1: U.S. Pat. No. 6,072,088

### SUMMARY OF INVENTION

### Technical Problem

An object of the present invention is to provide a novel method for producing 3-chloro-1,1,1,5,5,5-hexafluoro-2-pentene. Another object of the present invention is to provide: in relation to the above novel production method, a further novel production method and a novel compound, in particular, 1,1,3-trichloro-5,5,5-trifluoro-1,3-pentadiene as a novel compound; a method for producing 3-chloro-1,1,1,5,5,5-hexafluoro-2-pentene, comprising a step of reacting 1,1,3-trichloro-5,5,5-trifluoro-1,3-pentadiene with hydrogen fluoride; a method for producing 3-chloro-1,1,1,5,5,5-hexafluoro-2-pentene, comprising a step of reacting 1,1,3,5,5,5-hexachloro-1,3-pentadiene with hydrogen fluoride; and a method for producing 1,1,3,5,5,5-hexachloro-1,3-pentadiene from 1,1,1,3,3,5,5,5-octachloropentane.

### Solution to Problem

The present invention provides the following.
[1] A method for producing 3-chloro-1,1,1,5,5,5-hexafluoro-2-pentene, comprising a step of reacting 1,1,1,3,3,5,5,5-octachloropentane with hydrogen fluoride.
[2] The method for producing 3-chloro-1,1,1,5,5,5-hexafluoro-2-pentene according to [1], comprising a step of reacting 1,1,1,3,3,5,5,5-octachloropentane with hydrogen fluoride in the presence of a metal halide catalyst in a liquid phase.
[3] The method according to [2], wherein the metal halide catalyst is selected from an antimony halide catalyst, a tin halide catalyst, a titanium halide catalyst, a niobium halide catalyst, a tantalum halide catalyst, or a combination thereof.
[4] The method according to [2], wherein the metal halide catalyst is selected from antimony trichloride, antimony pentachloride, antimony trifluoride, antimony pentafluoride, tin tetrachloride, titanium tetrachloride, niobium pentafluoride, tantalum pentafluoride, or a combination thereof.
[5] The method according to any one of [2] to [4], wherein hydrogen fluoride is used in an amount-of-substance ratio of hydrogen fluoride to 1,1,1,3,3,5,5,5-octachloropentane of 1 to 20.
[6] The method according to any one of [2] to [4], wherein the amount of the metal halide catalyst is 0.1 to 20 mol % based on the amount of 1,1,1,3,3,5,5,5-octachloropentane.
[7] The method according to any one of [2] to [4], wherein the reaction is carried out at a temperature of 0° C. to 200° C.
[8] The method according to any one of [2] to [4], wherein hydrogen fluoride is used in a molar equivalent ratio of hydrogen fluoride to 1,1,1,3,3,5,5,5-octachloropentane of 1 to 20, the amount of the metal halide catalyst is 0.1 to 20 mol % based on the amount of 1,1,1,3,3,5,5,5-octachloropentane, and the reaction is carried out at a temperature of 0° C. to 200° C.
[9] The method for producing 3-chloro-1,1,1,5,5,5-hexafluoro-2-pentene according to [1], comprising a step of reacting 1,1,1,3,3,5,5,5-octachloropentane with hydrogen fluoride in the presence of a metal catalyst in a gas phase.
[10] The method according to [9], wherein the metal catalyst is selected from a zinc catalyst supported on alumina, chromium trifluoride, chromium trichloride, chromium (III) oxide, or a combination thereof.
[11] The method according to [9], wherein the metal catalyst is supported on a support.
[12] The method according to [11], wherein the support is selected from aluminum fluoride, alumina, activated carbon, zeolite, or a combination thereof.
[13] The method according to any one of [9] to [12], wherein hydrogen fluoride is used in an amount-of-substance ratio of hydrogen fluoride to 1,1,1,3,3,5,5,5-octachloropentane of 1 to 20.
[14] The method according to any one of [9] to [12], wherein the amount of the metal catalyst is 0.01 to 10 mol % based on the amount of 1,1,1,3,3,5,5,5-octachloropentane.
[15] The method according to any one of [9] to [12], wherein the reaction is carried out at a temperature of 100° C. to 450° C.
[16] The method according to any one of [9] to [12], wherein hydrogen fluoride is used in an amount-of-substance ratio of hydrogen fluoride to 1,1,1,3,3,5,5,5-octachloropentane of 1 to 20, the amount of the metal catalyst is 0.01 to 10 mol % based on the amount of 1,1,1,3,3,5,5,5-octachloropentane, and the reaction is carried out at a temperature of 100° C. to 450° C.
[17] 1,1,3-Trichloro-5,5,5-trifluoro-1,3-pentadiene.
[18] A method for producing 3-chloro-1,1,1,5,5,5-hexafluoro-2-pentene, comprising a step of reacting 1,1,3-trichloro-5,5,5-trifluoro-1,3-pentadiene with hydrogen fluoride.
[19] A method for producing 3-chloro-1,1,1,5,5,5-hexafluoro-2-pentene, comprising a step of reacting 1,1,3,5,5,5-hexachloro-1,3-pentadiene with hydrogen fluoride.
[20] The method for producing 3-chloro-1,1,1,5,5,5-hexafluoro-2-pentene according to [19], comprising a step of reacting 1,1,3,5,5,5-hexachloro-1,3-pentadiene with hydrogen fluoride in the presence of a metal halide catalyst in a liquid phase.
[21] The method for producing 3-chloro-1,1,1,5,5,5-hexafluoro-2-pentene according to [19], comprising a step of reacting 1,1,3,5,5,5-hexachloro-1,3-pentadiene with hydrogen fluoride in the presence of a metal catalyst in a gas phase.
[22] A method for producing 1,1,3,5,5,5-hexachloro-1,3-pentadiene, comprising contacting 1,1,1,3,3,5,5,5-octachloropentane with a metal catalyst at a temperature of 40 to 190° C.

### Advantageous Effects of Invention

According to the present invention, 3-chloro-1,1,1,5,5,5-hexafluoro-2-pentene can be efficiently produced in a one-step reaction from easily available 1,1,1,3,3,5,5,5-octachloropentane as a starting material. According to the present invention, also provided are: a novel compound, 1,1,3-trichloro-5,5,5-trifluoro-1,3-pentadiene; a method for producing 3-chloro-1,1,1,5,5,5-hexafluoro-2-pentene, comprising a step of reacting 1,1,3-trichloro-5,5,5-trifluoro-1,3-pentadiene with hydrogen fluoride; a method for producing 3-chloro-1,1,1,5,5,5-hexafluoro-2-pentene, comprising a step of reacting 1,1,3,5,5,5-hexachloro-1,3-pentadiene with hydrogen fluoride; and a method for producing 1,1,3,5,5,5-hexachloro-1,3-pentadiene from 1,1,1,3,3,5,5,5-octachloropentane.

### BRIEF DESCRIPTION OF DRAWINGS

FIG. 1 is a schematic view of a reaction system used in Examples.

### DESCRIPTION OF EMBODIMENTS

### [Effects]

According to the present invention, 3-chloro-1,1,1,5,5,5-hexafluoro-2-pentene (HCFO-1446) can be efficiently produced in a one-step reaction from easily available 1,1,1,3,3,5,5,5-octachloropentane as a starting material. According to the present invention, the method for producing HCFO-1446 can be carried out either as a liquid phase reaction or as a gas phase reaction, and the target product can be obtained in a one-step reaction in a yield of 60% or more. The method proposed in the prior art literature (PTL 1) is a method for producing HCFO-1446 in a two-step reaction involving use of 1,1,1,3,3,5,5,5-octafluoropentane as a starting material, the two-step reaction including replacing fluorine with chlorine. However, since the method requires the starting material, 3-chloro-1,1,1,5,5,5-hexafluoro-2-pentene, to be produced from other easily available materials, it requires the production process including substantially more than two steps. Therefore, the method for producing HCFO-1446 of the present invention has the remarkable effect of producing the target product in a high yield that is completely unexpected from the prior art literature, by a novel procedure including replacing chlorine in 1,1,1,3,3,5,5,5-octachloropentane with fluorine.

[Method for Producing HCFO-1446 from 1,1,1,3,3,5,5,5-Octachloropentane as a Starting Material]

In the present invention, the step of reacting 1,1,1,3,3,5,5,5-octachloropentane with hydrogen fluoride can be carried out either in a liquid phase reaction or in a gas phase reaction.

### (Liquid Phase Reaction)

The liquid phase reaction is carried out by reacting 1,1,1,3,3,5,5,5-octachloropentane with hydrogen fluoride in the presence of a metal halide catalyst in a liquid phase.

The starting material for the production, 1,1,1,3,3,5,5,5-octachloropentane can be easily produced by any known methods or is easily available as a reagent.

Examples of the metal halide catalyst include an antimony halide catalyst, a tin halide catalyst, a titanium halide catalyst, a niobium halide catalyst and a tantalum halide catalyst, and these can be used singly or in combination of two or more thereof. Examples of the antimony halide catalyst include antimony trichloride, antimony pentachloride, antimony trifluoride and antimony pentafluoride; examples of the tin halide catalyst include tin tetrachloride; examples of the titanium halide catalyst include titanium tetrachloride; examples of the niobium halide catalyst include niobium pentafluoride; and examples of the tantalum halide catalyst include tantalum pentafluoride; and these can be used singly or in combination of two or more thereof.

Hydrogen fluoride is used preferably in an amount-of-substance ratio (molar ratio) of hydrogen fluoride to 1,1,1,3,3,5,5,5-octachloropentane of 1 to 20, more preferably 3 to 10 and most preferably 5 to 8.

The amount of the metal halide catalyst is preferably 0.1 to 20 mol %, more preferably 1 to 10 mol % and most preferably 2 to 3 mol %, based on 100 mol % of the amount of 1,1,1,3,3,5,5,5-octachloropentane. In particular, when antimony chloride is used, halogen exchange of the catalyst itself occurs, thereby consuming hydrogen fluoride, and accordingly, the amount of the catalyst used is desirably smaller.

The reaction temperature is preferably 0° C. to 200° C., more preferably 25° C. to 100° C., and most preferably 50° C. to 100° C. If the reaction temperature is too low, the reaction will not proceed. If it is too high, it will cause tar formation and damage to a reactor due to corrosion from hydrogen fluoride, hydrogen chloride or the like.

In the liquid phase reaction, hydrogen fluoride can be liquefied and used as a solvent. Since the boiling point of hydrogen fluoride is about 20° C., it is necessary for carrying out the liquid phase reaction within the above temperature range that the reaction should be carried out under pressurized conditions, for example, by using an autoclave as a reaction vessel.

### (Gas Phase Reaction)

The gas phase reaction is carried out by reacting 1,1,1,3,3,5,5,5-octachloropentane with hydrogen fluoride in the presence of a metal catalyst in a gas phase.

The starting material for the production, 1,1,1,3,3,5,5,5-octachloropentane can be easily produced by any known methods or is easily available as a reagent.

The metal catalyst is preferably a compound containing at least one metal selected from the group consisting of aluminum, vanadium, chromium, titanium, magnesium, manganese, iron, cobalt, nickel, copper, zirconium, niobium, molybdenum, ruthenium, rhodium, palladium, silver, tin, zinc, lanthanum, tantalum and tungsten, and more preferably an oxide, halide or oxyhalide of such a metal. Each of these metal compounds may be supported on a support. By supporting the metal compound on the support, the catalyst can be formed into the desired shape and the catalyst concentration on the support can also be adjusted. Examples of the support include aluminum fluoride, alumina, activated carbon and zeolite. The catalyst is uniformly distributed on the support so that the concentration of the catalyst on the support is an appropriate catalyst concentration for a starting material, as described below. More preferred examples of the metal catalyst include a zinc catalyst supported on alumina, and a chromium catalyst such as chromium trifluoride, chromium trichloride or chromium (III) oxide, and these can be used singly or in combination of two or more thereof.

Hydrogen fluoride is used preferably in an amount-of-substance ratio (molar ratio) of hydrogen fluoride to 1,1,1,3,3,5,5,5-octachloropentane of 1 to 20, more preferably 3 to 10 and most preferably 5 to 8.

The amount of the metal catalyst is preferably 0.01 to 10 mol %, more preferably 0.1 to 7 mol % and most preferably 0.1 to 5 mol %, based on 100 mol % of the amount of 1,1,1,3,3,5,5,5-octachloropentane.

The reaction temperature is preferably 100° C. to 450° C., more preferably 150° C. to 300° C., and most preferably 190° C. to 250° C.

Since the boiling point of hydrogen fluoride is about 20° C., it can be easily vaporized and introduced into a reactor.

### [Carrier Gas]

In carrying out the present invention, a carrier gas is used for diluting a starting material gas and drying a reactor, for example. By flowing the carrier gas, substances such as a starting material, hydrogen fluoride, other additives and a reaction product can be moved within a reaction system while adjusting the concentrations thereof, particularly in a gas phase reaction. The carrier gas to be selected is a gas that does not react with such substances. Examples of the carrier gas include nitrogen and a noble gas (such as helium, neon or argon). When using a carrier gas, it is usually mixed with substances such as a starting material, hydrogen fluoride, other additives and a reaction product, such that the proportion of the carrier gas is 0 to 50%, more preferably 0 to 25%, and most preferably 0 to 10%, based on the total amount of the flowing substances.

### [Reaction System]

Examples of the material for the reaction system include corrosion-resistant metals such as stainless steel, Inconel, Monel, Hastelloy and nickel. Among these, nickel is preferred in view of corrosion resistance.

Examples of the reaction system for a gas phase reaction include a cylindrical tube equipped with a heater for adjusting the reaction temperature, packed with a catalyst having various shapes and configured to allow a starting material gas to flow from one end of the tube to the other. For the direction of flowing the starting material gas, it is preferable to gradually flow the starting material gas uniformly from top to bottom, in a case where the cylindrical tube packed with the catalyst is configured to extend vertically, and the reason for this is because gravity can be used to flow the starting material gradually. In a case where the starting material gas is flowed from bottom to top of the cylindrical tube configured to extend vertically, it is preferable to place a catalyst in the form of pellet with a large particle diameter at the bottom of the cylindrical tube and the catalyst in the form of powder with a small particle diameter at the top of the cylindrical tube, in terms of reaction efficiency.

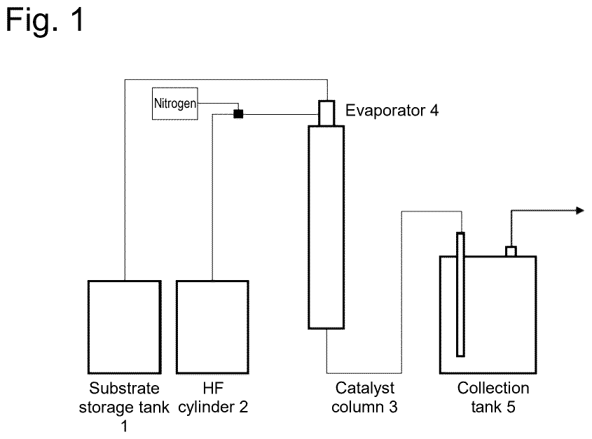

In FIG. 1, which schematically illustrates the specific example of the system to be used in the gas phase reaction, the system is composed of a substrate storage tank 1 for containing 1,1,1,3,3,5,5,5-octachloropentane; HF cylinder 2 for containing hydrogen fluoride; catalyst column 3 having a cylindrical shape, connected to the substrate storage tank and the HF cylinder by piping, and set up vertically; evaporator 4 positioned at the top of the catalyst column; and collection tank 5 located downstream of the catalyst column. 1,1,1,3,3,5,5,5-Octachloropentane (boiling point: 300° C.) in a liquid form is injected as it is by a syringe pump (not shown) into evaporator 4 heated to a temperature of its boiling point or more, and is vaporized in evaporator 4. Hydrogen fluoride is discharged as a gas from HF cylinder 2, mixed with a carrier gas (nitrogen gas) along the way of the piping, and introduced into evaporator 4. In evaporator 4, the vaporized 1,1,1,3,3,5,5,5-octachloropentane, hydrogen fluoride gas and carrier gas are mixed, and this mixed gas comes into contact with the catalyst as it moves from the top to the bottom of the catalyst column to promote the reaction. While the reaction gas that has passed through the bottom of the catalyst column passes through the collection tank filled with water, the reaction product, the unreacted starting material and hydrogen fluoride are collected in the collection tank, and only the carrier gas is discharged outside the reaction system.

[1,1,3-Trichloro-5,5,5-trifluoro-1,3-pentadiene]

As a result of carrying out the method of the present invention, it has been found that 1,1,3-trichloro-5,5,5-trifluoro-1,3-pentadiene (hereinafter sometimes referred to as “HCFO-2433”) is produced. HCFO-2433 is a novel compound produced for the first time by the method of the present invention. HCFO-2433 has, in its molecule, two C═C double bonds that are conjugated with each other, and each of the two C═C double bonds has a chlorine atom, a hydrogen atom and a trifluoromethyl group attached thereto. Accordingly, it can react with various reaction regents; and it can leave one C═C double bond in its molecule even after the reaction, so that it can be also used in another synthetic route. HCFO-2433 is extremely useful as a synthetic intermediate. In addition, HCFO-2433 can be used as a starting material in the method for producing HCFO-1446, as described below.

[Method for Producing HCFO-1446 from 1,1,3-trichloro-5,5,5-trifluoro-1,3-pentadiene as Starting Material]

HCFO-1446 can also be produced by reacting 1,1,3-trichloro-5,5,5-trifluoro-1,3-pentadiene with hydrogen fluoride. This reaction can be carried out either in a liquid phase reaction or in a gas phase reaction, similarly to the above-described method for producing HCFO-1446 by reacting 1,1,1,3,3,5,5,5-octachloropentane with hydrogen fluoride.

### (Liquid Phase Reaction)

The liquid phase reaction is carried out by reacting 1,1,3-trichloro-5,5,5-trifluoro-1,3-pentadiene with hydrogen fluoride in the presence of a metal halide catalyst in a liquid phase.

The starting material for the production, 1,1,3-trichloro-5,5,5-trifluoro-1,3-pentadiene has been produced for the first time in the method for producing HCFO-1446 of the present invention.

Examples of the metal halide catalyst include an antimony halide catalyst, a tin halide catalyst, a titanium halide catalyst, a niobium halide catalyst and a tantalum halide catalyst, and these can be used singly or in combination of two or more thereof. Examples of the antimony halide catalyst include antimony trichloride, antimony pentachloride, antimony trifluoride and antimony pentafluoride; examples of the tin halide catalyst include tin tetrachloride; examples of the titanium halide catalyst include titanium tetrachloride; examples of the niobium halide catalyst include niobium pentafluoride; and examples of the tantalum halide catalyst include tantalum pentafluoride; and these can be used singly or in combination of two or more thereof.

Hydrogen fluoride is used preferably in an amount-of-substance ratio (molar ratio) of hydrogen fluoride to 1,1,3-trichloro-5,5,5-trifluoro-1,3-pentadiene of 1 to 20, more preferably 3 to 10 and most preferably 5 to 8.

The amount of the metal halide catalyst is preferably 0.1 to 20 mol %, more preferably 1 to 10 mol % and most preferably 2 to 3 mol %, based on 100 mol % of the amount of 1,1,3-trichloro-5,5,5-trifluoro-1,3-pentadiene. In particular, when antimony chloride is used, halogen exchange of the catalyst itself occurs, thereby consuming hydrogen fluoride, and accordingly, the amount of the catalyst used is desirably smaller.

The reaction temperature is preferably 0° C. to 200° C., more preferably 25° C. to 100° C., and most preferably 50° C. to 100° C. If the reaction temperature is too low, the reaction will not proceed. If it is too high, it will cause tar formation and damage to a reactor due to corrosion from hydrogen fluoride, hydrogen chloride or the like.

In the liquid phase reaction, hydrogen fluoride can be liquefied and used as a solvent. Since the boiling point of hydrogen fluoride is about 20° C., it is necessary for carrying out the liquid phase reaction within the above temperature range that the reaction should be carried out under pressurized conditions, for example, by using an autoclave as a reaction vessel.

### (Gas Phase Reaction)

The gas phase reaction is carried out by reacting 1,1,3-trichloro-5,5,5-trifluoro-1,3-pentadiene with hydrogen fluoride in the presence of a metal catalyst in a gas phase.

The starting material for the production, 1,1,3-trichloro-5,5,5-trifluoro-1,3-pentadiene has been produced for the first time in the method for producing HCFO-1446 of the present invention.

The metal catalyst is preferably a compound containing at least one metal selected from the group consisting of aluminum, vanadium, chromium, titanium, magnesium, manganese, iron, cobalt, nickel, copper, zirconium, niobium, molybdenum, ruthenium, rhodium, palladium, silver, tin, zinc, lanthanum, tantalum and tungsten, and more preferably an oxide, halide or oxyhalide of such a metal. Each of these metal compounds may be supported on a support. By supporting the metal compound on the support, the catalyst can be formed into the desired shape and the catalyst concentration on the support can also be adjusted. Examples of the support include aluminum fluoride, alumina, activated carbon and zeolite. The catalyst is uniformly distributed on the support so that the concentration of the catalyst on the support is an appropriate catalyst concentration for a starting material, as described below. More preferred examples of the metal catalyst include a zinc catalyst supported on alumina, a chromium catalyst such as chromium trifluoride, chromium trichloride or chromium (III) oxide, and these can be used singly or in combination of two or more thereof.

Hydrogen fluoride is used preferably in an amount-of-substance ratio (molar ratio) of hydrogen fluoride to 1,1,3-trichloro-5,5,5-trifluoro-1,3-pentadiene of 1 to 20, more preferably 3 to 10 and most preferably 5 to 8.

The amount of the metal catalyst is preferably 0.01 to 10 mol %, more preferably 0.1 to 7 mol % and most preferably 0.1 to 5 mol %, based on 100 mol % of the amount of 1,1,3-trichloro-5,5,5-trifluoro-1,3-pentadiene.

The reaction temperature is preferably 100° C. to 450° C., more preferably 150° C. to 300° C., and most preferably 190° C. to 250° C.

Since the boiling point of hydrogen fluoride is about 20° C., it can be easily vaporized and introduced into a reactor.

[Method for Producing HCFO-1446 from 1,1,3,5,5,5-hexachloro-1,3-pentadiene as a Starting Material]

HCFO-1446 can also be produced by reacting 1,1,3,5,5,5-hexachloro-1,3-pentadiene with hydrogen fluoride. This reaction can be carried out either in a liquid phase reaction or in a gas phase reaction, similarly to the above-described method for producing HCFO-1446 by reacting 1,1,1,3,3,5,5,5-octachloropentane with hydrogen fluoride.

### (Liquid Phase Reaction)

The liquid phase reaction is carried out by reacting 1,1,3,5,5,5-hexachloro-1,3-pentadiene with hydrogen fluoride in the presence of a metal halide catalyst in a liquid phase.

The starting material for the production, 1,1,3,5,5,5-hexachloro-1,3-pentadiene can be easily produced by any known methods or is easily available as a reagent.

Examples of the metal halide catalyst include an antimony halide catalyst, a tin halide catalyst, a titanium halide catalyst, a niobium halide catalyst and a tantalum halide catalyst, and these can be used singly or in combination of two or more thereof. Examples of the antimony halide catalyst include antimony trichloride, antimony pentachloride, antimony trifluoride and antimony pentafluoride; examples of the tin halide catalyst include tin tetrachloride; examples of the titanium halide catalyst include titanium tetrachloride; examples of the niobium halide catalyst include niobium pentafluoride; and examples of the tantalum halide catalyst include tantalum pentafluoride; and these can be used singly or in combination of two or more thereof.

Hydrogen fluoride is used preferably in an amount-of-substance ratio (molar ratio) of hydrogen fluoride to 1,1,3,5,5,5-hexachloro-1,3-pentadiene of 1 to 20, more preferably 3 to 10 and most preferably 5 to 8.

The amount of the metal halide catalyst is preferably 0.1 to 20 mol %, more preferably 1 to 10 mol % and most preferably 2 to 3 mol %, based on 100 mol % of the amount of 1,1,3,5,5,5-hexachloro-1,3-pentadiene. In particular, when antimony chloride is used, halogen exchange of the catalyst itself occurs, thereby consuming hydrogen fluoride, and accordingly, the amount of the catalyst used is desirably smaller.

The reaction temperature is preferably 0° C. to 200° C., more preferably 25° C. to 100° C., and most preferably 50° C. to 100° C. If the reaction temperature is too low, the reaction will not proceed. If it is too high, it will cause tar formation and damage to a reactor due to corrosion from hydrogen fluoride, hydrogen chloride or the like.

In the liquid phase reaction, hydrogen fluoride can be liquefied and used as a solvent. Since the boiling point of hydrogen fluoride is about 20° C., it is necessary for carrying out the liquid phase reaction within the above temperature range that the reaction should be carried out under pressurized conditions, for example, by using an autoclave as a reaction vessel.

### (Gas Phase Reaction)

The gas phase reaction is carried out by reacting 1,1,3,5,5,5-hexachloro-1,3-pentadiene with hydrogen fluoride in the presence of a metal catalyst in a gas phase.

The starting material for the production, 1,1,3,5,5,5-hexachloro-1,3-pentadiene can be easily produced by any known methods or is easily available as a reagent.

The metal catalyst is preferably a compound containing at least one metal selected from the group consisting of aluminum, vanadium, chromium, titanium, magnesium, manganese, iron, cobalt, nickel, copper, zirconium, niobium, molybdenum, ruthenium, rhodium, palladium, silver, tin, zinc, lanthanum, tantalum and tungsten, and more preferably an oxide, halide or oxyhalide of such a metal. Each of these metal compounds may be supported on a support. By supporting the metal compound on the support, the catalyst can be formed into the desired shape and the catalyst concentration on the support can also be adjusted. Examples of the support include aluminum fluoride, alumina, activated carbon and zeolite. The catalyst is uniformly distributed on the support so that the concentration of the catalyst on the support is an appropriate catalyst concentration for a starting material, as described below. More preferred examples of the metal catalyst include a zinc catalyst supported on alumina, a chromium catalyst such as chromium trifluoride, chromium trichloride or chromium (III) oxide, and these can be used singly or in combination of two or more thereof.

Hydrogen fluoride is used preferably in an amount-of-substance ratio (molar ratio) of hydrogen fluoride to 1,1,3,5,5,5-hexachloro-1,3-pentadiene of 1 to 20, more preferably 3 to 10 and most preferably 5 to 8.

The amount of the metal catalyst is preferably 0.01 to 10 mol %, more preferably 0.1 to 7 mol % and most preferably 0.1 to 5 mol %, based on 100 mol % of the amount of 1,1,3,5,5,5-hexachloro-1,3-pentadiene.

The reaction temperature is preferably 100° C. to 450° C., more preferably 150° C. to 300° C., and most preferably 190° C. to 250° C.

Since the boiling point of hydrogen fluoride is about 20° C., it can be easily vaporized and introduced into a reactor.

[Method for Producing 1,1,3,5,5,5-hexachloro-1,3-pentadiene] 1,1,3,5,5,5-Hexachloro-1,3-pentadiene can be Produced by Contacting 1,1,1,3,3,5,5,5-octachloropentane with a Metal Catalyst at a Temperature of 40 to 190° C.

The metal catalyst to be used for the reaction is preferably a compound containing at least one metal selected from the group consisting of aluminum, vanadium, chromium, titanium, magnesium, manganese, iron, cobalt, nickel, copper, zirconium, niobium, molybdenum, ruthenium, rhodium, palladium, silver, tin, zinc, lanthanum, tantalum and tungsten, and more preferably an oxide, halide or oxyhalide of such a metal. Each of these metal compounds may be supported on a support. By supporting the metal compound on the support, the catalyst can be formed into the desired shape and the catalyst concentration on the support can also be adjusted. Examples of the support include alumina, activated carbon and zeolite. The catalyst is uniformly distributed on the support so that the concentration of the catalyst on the support is an appropriate catalyst concentration for a starting material, as described below. More preferred examples of the metal catalyst include iron (III) chloride and aluminum (III) chloride, and these can be used singly or in combination of two or more thereof.

The reaction temperature is preferably 40° C. to 190° C., more preferably 60° C. to 120° C., and most preferably 70° C. to 100° C.

### EXAMPLES

### Liquid Phase Halogen Exchange Reaction of 1,1,1,3,3,5,5,5-octachloropentane with HF by SbCl₅ Catalyst

### Example 1-1

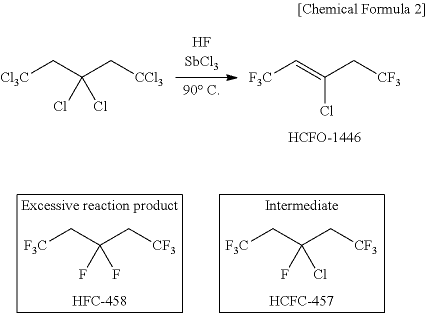

A 313 g portion (0.83 mol) of 1,1,1,3,3,5,5,5-octachloropentane (sometimes abbreviated as “8Cl compound”) and 7.47 g (25 mmol, 3 mol %) of SbCl₅ were placed in a 50 mL SUS316 autoclave equipped with a thermometer, a pressure gauge and a needle valve, and a stirrer, and 100 g (5.00 mol, 6.02× 102 mol %) of anhydrous HF was then added thereto, followed by heating to 90° C. The reaction was allowed to continue by maintaining the temperature at 90° C. for 4.5 hours, after which the autoclave was cooled in an ice bath to slowly release the HCl generated in the system into a water trap. Next, the autoclave was heated to 40° C. to release the unreacted HF into the water trap. After cooling, the reaction liquid was poured into water and was subjected to liquid separation to obtain 191 g of an organic layer. Quantitative determination of the organic layer by NMR showed that: the composition of the organic layer was 75% of 3-chloro-1,1,1,5,5,5-hexafluoro-2-pentene (E/Z=19/81) and 2.0% of 3-chloro-1,1,1,3,5,5,5-heptafluoropentane, and 3-chloro-1,1,1,5,5,5-hexafluoro-2-pentene was obtained in a quantitative yield of 75%.

The above production process was carried out a total of eight times to obtain 1,451 g of a crude product. This crude product was subjected to distillation under atmospheric pressure using a glass still equipped with a condenser to obtain 673 g of 3-chloro-1,1,1,5,5,5-hexafluoro-2-pentene with a GC purity of >99%.

### Examples 1-2 and 1-3 and Comparative Examples 1-1 and 1-2

The reaction was carried out in the same manner as in Example 1-1, except that the molar equivalent of hydrogen fluoride relative to 1,1,1,3,3,5,5,5-octachloropentane, the concentration of the antimony catalyst and the reaction time were changed. The results are shown in Table 1. In Table 1, “N.D.” indicates that it was impossible to calculate the conversion rate, and “Trace” indicates that the amount was a trace amount.

TABLE 1

| | HF | | | | | | |
|---|---|---|---|---|---|---|---|
| | amount-of- | SbCl5 | | | Yield | | |
| | substance | mol %/ | Reaction | Conversion | HCFO- | HCFC- | HFC- |
| | ratio/to | relative to | time | rate | 1446 | 457 | 458 |
| Example | substrate | substrate | H | % | % | % | % |
| 1-1 | 6.0 | 3 | 4.5 | >99 | 75 | 2 | 0 |
| 1-2 | 6.0 | 2 | 3.0 | >99 | 62 | 2 | 0 |
| 1-3 | 7.0 | 2 | 2.0 | >99 | 63 | 1 | 0 |
| Comparative | 8.5 | 1 | 5.5 | N.D. | 12 | Trace | 0 |
| Example 1-1 | | | | | | | |
| Comparative | 9.5 | 5 | 7.5 | >99 | 3 | 8 | 70 |
| Example 1-2 | | | | | | | |

The results of Table 1 shows that the yield of 3-chloro-1,1,1,5,5,5-hexafluoro-2-pentene exceeded 60% when the amount-of-substance ratio of hydrogen fluoride to 1,1,1,3,3,5,5,5-octachloropentane was 6.0 to 7.0 with a concentration of antimony pentachloride of 2 to 3 mol % and a reaction time of 2.0 to 4.5 hours. It was found that under conditions that the fluorination reaction easily occurs, such as a high molar equivalent of hydrogen fluoride, a high concentration of antimony pentachloride and a long reaction time, the main product was 1,1,1,3,3,5,5,5-octafluoropentane (8 F-compound: HFC-458), in which all of the chlorines of the starting material 8Cl-compound were replaced with fluorine, resulting in a decrease in the yield of the target product. On the other hand, it was found that in a case where the concentration of the catalyst antimony pentachloride was low, it was impossible to control the reaction even when the molar equivalent of hydrogen fluoride is high, and that it resulted in not only a decreased yield of the target product but also a wide variety of products.

Gas Phase Halogen Exchange Reaction of 1,1,1,3,3,5,5,5-octachloropentane with HF and CrF₃/AlF₃ catalyst

### Preparation of 5.1 Mol % CrF₃/AlF₃ Catalyst

A 308 ml portion of ultrapure water was placed in a 500 ml flask, 63.15 g (0.237 mol) of CrCl₃·6H₂O was added thereto, and the mixture was stirred at room temperature to dissolve it completely. Next, 212.60 g (2.085 mol) of spherical γ-Al₂O₃ having a diameter of 2 to 4 mm was added thereto, and the resultant was allowed to stand for 1 hour. Thereafter, water was distilled off by gradually reducing the pressure from 40 hPa to 5 hPa at 40° C. to 60° C. with stirring to prepare 251 g of 10.2 mol % CrCl₃/Al₂O₃ catalyst. A 119.4 g portion of the 10.2 mol % CrCl₃/Al₂O₃ catalyst was packed into a nickel catalyst column having an inner diameter of 20 mm×400 mm, and the catalyst column was supplied with HF at a flow rate of 286 SCCM and with nitrogen at a flow rate of 100 SCCM. The catalyst was activated while the catalyst column was heated to 200° C. The catalyst was activated by gradually increasing the heating temperature of the catalyst column to 400° C. As the activation of the catalyst progressed, acidic water was discharged from the catalyst column. The activation was terminated when the acidic water ceased to be discharged. The concentration of the catalyst (5.1 mol %) was expressed in terms of the ratio of the amount-of-substance of CrF₃ to the total amount-of-substance of catalyst, as shown in the following calculation formula.

Calculation formula: amount-of-substance of CrF₃/(amount-of-substance of CrF₃+amount-of-substance of AlF₃)

### Example 2-1

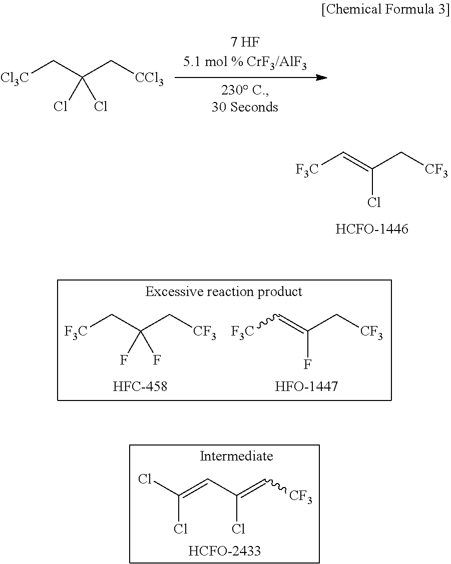

In the system shown in FIG. 1 including as a reactor a catalyst column (φ 22.4×400 mm, 157 ml) packed with 5.1 mol % CrF₃/AlF₃ catalyst, an HF line was heated to 60° C., an evaporator was heated to an external temperature of 180° C. and the catalyst column was heated to an internal temperature of 230° C. While supplying the catalyst column with nitrogen at 15.7 SCCM, a main valve of a 300 ml HF cylinder heated to 45° C. was opened to allow HF to flow at a flow rate of 262 SCCM for 15 minutes. Thereafter, while allowing HF to flow at 262 SCCM and nitrogen at 15 SCCM, the catalyst column was supplied with 1,1,1,3,3,5,5,5-octachloropentane at a rate of 20.9 ml/h (0.57 g/min, 37 SCCM) by a syringe pump and was aerated to react them. The product was collected in a water trap cooled to 0° C. The catalyst column was supplied with 52.5 g (0.15 mol) of 1,1,1,3,3,5,5,5-octachloropentane. After the supply of 1,1,1,3,3,5,5,5-octachloropentane was stopped, HF was allowed to flow at 262 SCCM for 30 minutes. Thereafter, the gas to be introduced was switched to nitrogen, the entire system was purged with nitrogen for 1 hour, and the organic layer was then recovered.

The recovered organic layer was neutralized and washed with 10 ml of 5% aqueous sodium bicarbonate solution to obtain 30.3 g of an organic layer. Quantitative determination of the organic layer by NMR showed that: the composition of the organic layer was 84.1% of 3-chloro-1,1,1,5,5,5-hexafluoro-2-pentene (E/Z=39/61), 6.0% of 1,1,1,3,5,5,5-heptafluoro-2-pentene (E/Z=43/57), 7.9% of 1,1,3-trichloro-5,5,5-trifluoro-1,3-pentadiene (E/Z=52/48) and 2.0% of 1,1,1,3,3,5,5,5-octafluoropentane, and 3-chloro-1,1,1,5,5,5-hexafluoro-2-pentene was obtained in a quantitative yield of 79%.

The above production process was repeated to obtain 378 g of a crude product containing 212 g of 3-chloro-1,1,1,5,5,5-hexafluoro-2-pentene. This crude product was subjected to distillation under atmospheric pressure using a glass still equipped with a condenser to obtain 189 g of 3-chloro-1,1,1,5,5,5-hexafluoro-2-pentene with a GC purity of >95% in an isolated yield of 71%.

The by-product obtained above, 1,1,3-trichloro-5,5,5-trifluoro-1,3-pentadiene, is a novel substance. The compound data is listed below.

### 1,1,3-trichloro-5,5,5-trifluoro-1,3-pentadiene

Boiling point (E/Z isomer mixture): 123 to 136° C. @ 1 atm (actual found value: 56 to 70° C. @ 55 hPa)
Density (E/Z isomer mixture): 1.52 g/cm³
(E)-Isomer:
¹H-NMR δ=6.30 (dq, J₁=0.8 Hz, J₂=7.2 Hz, 1H, CF₃—CH═), 6.41 (t, J=0.8 Hz, 1H, CH═CCl₂),
¹⁹F-NMR δ=−59.38 (d, J=7.5 Hz, 3 F)
GC-MS: m/z=223.9, 225.9, 227.9, calculated value for C₅H₂Cl₃F₃: 225.42
(Z)-Isomer:
¹H-NMR δ=6.06 (dq, J₁=2.0 Hz, J₂=7.6 Hz, 1H, CF₃—CH═), 6.60 (d, J=2.0 Hz, 1H, CH═CCl₂),
¹⁹F-NMR δ=−59.60 (dd, J₁=2.3 Hz, J₂=7.9 Hz, 3 F)
GC-MS: m/z=223.9, 225.9, 227.9, calculated value for C₅H₂Cl₃F₃: 225.42

### Examples 2-2 and 2-3

The reaction was carried out in the same manner as in Example 2-1, except that the reaction temperature was changed. The results are shown in Table 2.

TABLE 2

| Example | | | 2-1 | 2-2 | 2-3 |
|---|---|---|---|---|---|
| Amount of HF | | amount-of-substance | 7.1 | 7.1 | 7.1 |
| | | ratio / to substrate | | | |
| Reaction temperature | | ° C. | 230 | 250 | 200 |
| Contact time | | second | 30 | 30 | 30 |
| Composition of | HCFO-1446 | % (E/Z) | 84.10 (40/60) | 81.44 (41/59) | 74.93 (34/66) |
| Organic layer | HFO-1447 | % (E/Z) | 6.02 (43/57) | 6.94 (37/63) | 5.47 (41/59) |
| | HCFO-2433 | % (E/Z) | 7.91 (52/48) | 9.78 (50/50) | 17.56 (48/52) |
| | HFC-458 | % | 1.97 | 1.84 | 2.04 |

| | | | | |
|---|---|---|---|---|
| Recovery rate | % | 94 | 91 | 87 |
| Conversion rate | % | >99 | >99 | >99 |
| Selectivity | % | 84 | 81 | 75 |
| Yield | % | 79 | 74 | 65 |

The results of Table 2 shows that the yield of 3-chloro-1,1,1,5,5,5-hexafluoro-2-pentene exceeded 60% when the amount-of-substance ratio of hydrogen fluoride to 1,1,1,3,3,5,5,5-octachloropentane was 5 to 8 with a reaction temperature of 200 to 250° C.

Gas Phase Halogen Exchange Reaction of 1,1,3-trichloro-5,5,5-trifluoro-1,3-pentadiene with HF and CrF₃/AlF₃ Catalyst

### Example 3

A method for producing 3-chloro-1,1,1,5,5,5-hexafluoro-2-pentene from the novel compound (1,1,3-trichloro-5,5,5-trifluoro-1,3-pentadiene) generated as a by-product in Example 2-1 was investigated.

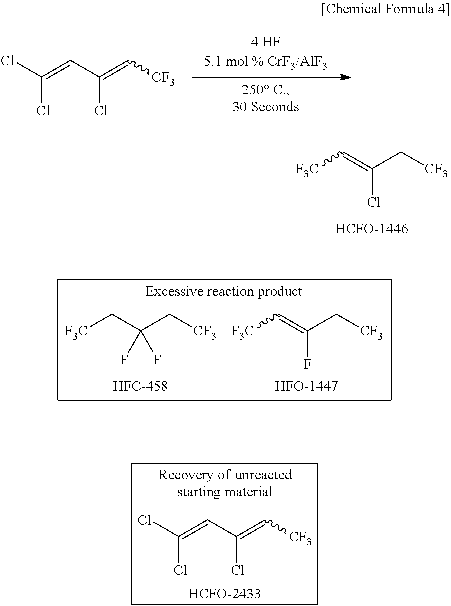

In the system shown in FIG. 1 including as a reactor a catalyst column (q 22.4×400 mm, 157 ml) packed with 5.1 mol % CrF₃/AlF₃ catalyst, an HF line was heated to 60° C., an evaporator was heated to an external temperature of 180° C. and the catalyst column was heated to an internal temperature of 250° C. While supplying the catalyst column with nitrogen (N₂) at 15.7 SCCM from the evaporator, a main valve of a 300 ml HF cylinder heated to 50° C. was opened to allow HF to flow at a flow rate of 239 SCCM for 30 minutes. Thereafter, while allowing HF to flow at 239 SCCM and N₂ at 15 SCCM, the catalyst column was supplied with 1,1,3-trichloro-5,5,5-trifluoro-1,3-pentadiene at a rate of 24.0 ml/h (3.60 g/min, 60 SCCM) by a syringe pump and was aerated to react them. The product was collected in a water trap cooled to 0° C. The catalyst column was supplied with 97.6 g (0.43 mol) of 1,1,3-trichloro-5,5,5-trifluoro-1,3-pentadiene. After the supply of 1,1,3-trichloro-5,5,5-trifluoro-1,3-pentadiene was stopped, HF was allowed to flow at 239 SCCM for 30 minutes. Thereafter, the gas to be introduced was switched to nitrogen, the entire system was purged with nitrogen for 1 hour, and the organic layer was then recovered.

The recovered organic layer was neutralized and washed with 10 ml of 5% aqueous sodium bicarbonate solution to obtain 89.0 g of an organic layer. Quantitative determination of the organic layer by NMR showed that: the composition of the organic layer was 75.4% of 3-chloro-1,1,1,5,5,5-hexafluoro-2-pentene (E/Z=39/61), 10.2% of 1,1,1,3,5,5,5-heptafluoro-2-pentene (E/Z=42/58), 12.8% of 1,1,3-trichloro-5,5,5-trifluoro-1,3-pentadiene (E/Z=51/49) and 1.6% of 1,1,1,3,3,5,5,5-octafluoropentane, and 3-chloro-1,1,1,5,5,5-hexafluoro-2-pentene was obtained in a quantitative yield of 72%.

### Synthesis of 1,1,3,5,5,5-hexachloro-1,3-pentadiene

### Example 6

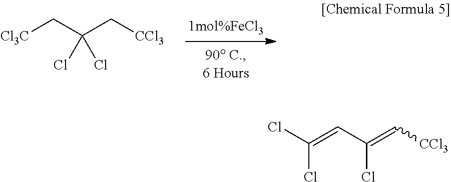

A 1,045 g portion (3.0 mol) of 1,1,1,3,3,5,5,5-octachloropentane (8Cl compound) was placed in a 1 L three-neck flask equipped with a Dimroth condenser with chiller circulated at 5° C. and a thermometer. At room temperature, 5.0 g of FeCl₃ (0.03 mol, 1 mol % (based on the number of moles of 8Cl compound) was added, followed by increasing the temperature to 90° C., and the reaction was carried out for 6 hours. The HCl generated as a by-product during the reaction was absorbed in a water trap. After the reaction for 6 hours, the reaction liquid was allowed to cool to room temperature, and poured into a water trap to stop the reaction. The resultant was subjected to liquid separation to recover an organic layer. The organic layer was dried over 20 g of Na₂SO₄, and then filtered to obtain 810 g of a crude product. The obtained crude product was distilled under reduced pressure at <1 hPa using a distillation column (the number of theoretical plates: about 2.7) equipped with a φ 19×300 mm Beagle fractionating tube, and 776 g of 1,1,3,5,5,5-hexachloro-1,3-pentadiene with a GC purity of 98.4% was obtained from the fraction at a column top temperature of 80 to 88° C., in an isolated yield of 92%.

Liquid Phase HF Halogen Exchange Reaction of 1,1,3,5,5,5-hexachloro-1,3-pentadiene with SbCl₅ Catalyst

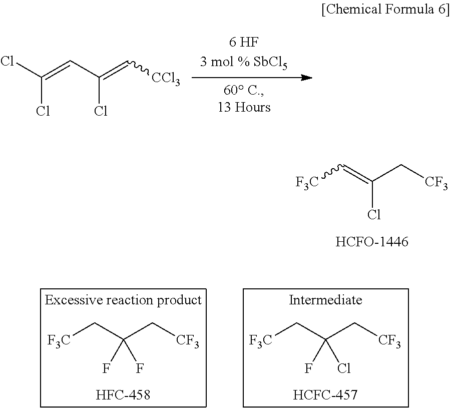

A 66.76 g portion (0.24 mol) of 1,1,3,5,5,5-hexachloro-1,3-pentadiene and 2.15 g (7.2 mmol, 3 mol %) of SbCl₅ were placed in a 100 mL SUS316 autoclave equipped with a thermometer, a pressure gauge and a needle valve, and a stirrer, and 29.11 g (1.46 mol, 599 mol %) of anhydrous HF was then added thereto, followed by heating to 60° C. After heating for 13 hours, the autoclave was allowed to cool to room temperature to slowly release the HCl generated in the system into a water trap. After cooling the autoclave with ice water, it was opened, and the reaction liquid was poured into water. The resultant was subjected to liquid separation to obtain 46.32 g of an organic layer. Quantitative determination of the organic layer by NMR showed that: the composition of the organic layer was 96% of 3-chloro-1,1,1,5,5,5-hexafluoro-2-pentene (E/Z=27/73) and 1.5% of 3-chloro-1,1,1,3,5,5,5-heptafluoropentane. 3-Chloro-1,1,1,5,5,5-hexafluoro-2-pentene was obtained from the obtained organic layer in a quantitative yield of 86%.

The above production process was repeated to obtain 142 g of a crude product containing 138 g of 3-chloro-1,1,1,5,5,5-hexafluoro-2-pentene. This crude product was subjected to distillation under atmospheric pressure using a glass still equipped with a condenser to obtain 128 g of 3-chloro-1,1,1,5,5,5-hexafluoro-2-pentene with a GC purity of >99% in an isolated yield of 80%.

Gas Phase HF Halogen Exchange Reaction of 1,1,3,5,5,5-hexachloro-1,3-pentadiene by CrF₃/AlF₃ Catalyst

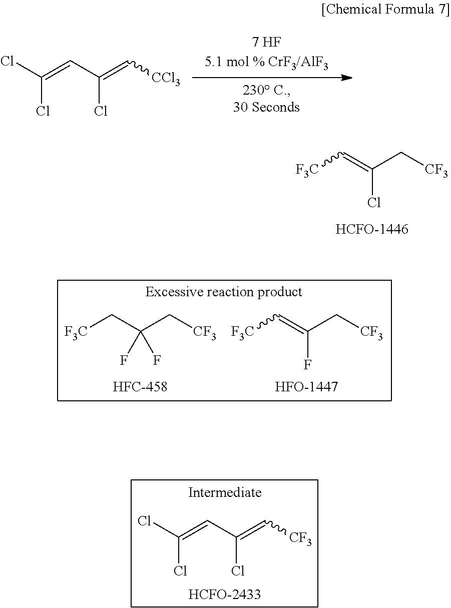

In the system shown in FIG. 1 including as a reactor a catalyst column (q 22.4×400 mm, 157 ml) packed with 5.1 mol % CrF₃/AlF₃ catalyst, an HF line was heated to 60° C., an evaporator was heated to an external temperature of 180° C. and the catalyst column was heated to an internal temperature of 230° C., and a main valve of a 300 ml HF cylinder heated to 45° C. was opened to allow HF to flow at a flow rate of 276 SCCM for 30 minutes. Thereafter, while allowing HF to flow at 276 SCCM, the catalyst column was supplied with 1,1,3,5,5,5-hexachloro-1,3-pentadiene at a rate of 17.8 ml/h (0.47 g/min, 37 SCCM) by a syringe pump and was aerated to react them. The product was collected in a water trap cooled to 0° C. The catalyst column was supplied with 57.7 g (0.21 mol) of 1,1,3,5,5,5-hexachloro-1,3-pentadiene. After the supply of 1,1,3,5,5,5-hexachloro-1,3-pentadiene was stopped, HF was allowed to flow at 276 SCCM for 30 minutes. Thereafter, the gas to be introduced was switched to nitrogen, the entire system was purged with nitrogen for 1 hour, and the organic layer was then recovered. The recovered organic layer was neutralized and washed with 10 ml of 5% aqueous sodium bicarbonate solution to obtain 41.9 g of an organic layer. Quantitative determination of the organic layer by NMR showed that: the composition of the organic layer was 83.5% of 3-chloro-1,1,1,5,5,5-hexafluoro-2-pentene (E/Z=42/58), 6.1% of 1,1,1,3,5,5,5-heptafluoro-2-pentene (E/Z=43/57), 9.1% of 1,1,3-trichloro-5,5,5-trifluoro-1,3-pentadiene (E/Z=52/48) and 1.4% of 1,1,1,3,3,5,5,5-octafluoropentane, and 3-chloro-1,1,1,5,5,5-hexafluoro-2-pentene was obtained in a quantitative yield of 78%.

1. A method for producing 3-chloro-1,1,1,5,5,5-hexafluoro-2-pentene, comprising a step of reacting 1,1,1,3,3,5,5,5-octachloropentane with hydrogen fluoride.

2. The method for producing 3-chloro-1,1,1,5,5,5-hexafluoro-2-pentene according to claim 1, comprising a step of reacting 1,1,1,3,3,5,5,5-octachloropentane with hydrogen fluoride in the presence of a metal halide catalyst in a liquid phase.

3. The method according to claim 2, wherein the metal halide catalyst is selected from an antimony halide catalyst, a tin halide catalyst, a titanium halide catalyst, a niobium halide catalyst, a tantalum halide catalyst, or a combination thereof.

4. The method according to claim 2, wherein the metal halide catalyst is selected from antimony trichloride, antimony pentachloride, antimony trifluoride, antimony pentafluoride, tin tetrachloride, titanium tetrachloride, niobium pentafluoride, tantalum pentafluoride, or a combination thereof.

5. The method according to claim 2, wherein hydrogen fluoride is used in an amount-of-substance ratio of hydrogen fluoride to 1,1,1,3,3,5,5,5-octachloropentane of 1 to 20.

6. The method according to claim 2, wherein the amount of the metal halide catalyst is 0.1 to 20 mol % based on the amount of 1,1,1,3,3,5,5,5-octachloropentane.

7. The method according to claim 2, wherein the reaction is carried out at a temperature of 0° C. to 200° C.

8. The method according to claim 2, wherein hydrogen fluoride is used in a molar equivalent ratio of hydrogen fluoride to 1,1,1,3,3,5,5,5-octachloropentane of 1 to 20, the amount of the metal halide catalyst is 0.1 to 20 mol % based on the amount of 1,1,1,3,3,5,5,5-octachloropentane, and the reaction is carried out at a temperature of 0° C. to 200° C.

9. The method for producing 3-chloro-1,1,1,5,5,5-hexafluoro-2-pentene according to claim 1, comprising a step of reacting 1,1,1,3,3,5,5,5-octachloropentane with hydrogen fluoride in the presence of a metal catalyst in a gas phase.

10. The method according to claim 9, wherein the metal catalyst is selected from a zinc catalyst supported on alumina, chromium trifluoride, chromium trichloride, chromium (III) oxide, or a combination thereof.

11. The method according to claim 9, wherein the metal catalyst is supported on a support.

12. The method according to claim 11, wherein the support is selected from aluminum fluoride, alumina, activated carbon, zeolite, or a combination thereof.

13. The method according to claim 9, wherein hydrogen fluoride is used in an amount-of-substance ratio of hydrogen fluoride to 1,1,1,3,3,5,5,5-octachloropentane of 1 to 20.

14. The method according to claim 9, wherein the amount of the metal catalyst is 0.01 to 10 mol % based on the amount of 1,1,1,3,3,5,5,5-octachloropentane.

15. The method according to claim 9, wherein the reaction is carried out at a temperature of 100° C. to 450° C.

16. The method according to claim 9, wherein hydrogen fluoride is used in an amount-of-substance ratio of hydrogen fluoride to 1,1,1,3,3,5,5,5-octachloropentane of 1 to 20, the amount of the metal catalyst is 0.01 to 10 mol % based on the amount of 1,1,1,3,3,5,5,5-octachloropentane, and the reaction is carried out at a temperature of 100° C. to 450° C.

17. 1,1,3-Trichloro-5,5,5-trifluoro-1,3-pentadiene.

18. A method for producing 3-chloro-1,1,1,5,5,5-hexafluoro-2-pentene, comprising a step of reacting 1,1,3-trichloro-5,5,5-trifluoro-1,3-pentadiene with hydrogen fluoride.

19. A method for producing 3-chloro-1,1,1,5,5,5-hexafluoro-2-pentene, comprising a step of reacting 1,1,3,5,5,5-hexachloro-1,3-pentadiene with hydrogen fluoride.

20. The method for producing 3-chloro-1,1,1,5,5,5-hexafluoro-2-pentene according to claim 19, comprising a step of reacting 1,1,3,5,5,5-hexachloro-1,3-pentadiene with hydrogen fluoride in the presence of a metal halide catalyst in a liquid phase.

21. The method for producing 3-chloro-1,1,1,5,5,5-hexafluoro-2-pentene according to claim 19, comprising a step of reacting 1,1,3,5,5,5-hexachloro-1,3-pentadiene with hydrogen fluoride in the presence of a metal catalyst in a gas phase.

22. A method for producing 1,1,3,5,5,5-hexachloro-1,3-pentadiene, comprising contacting 1,1,1,3,3,5,5,5-octachloropentane with a metal catalyst at a temperature of 40 to 190° C.
